(19) United States
(12) Patent Application Publication
(10) Pub. No.: US 2025/0236837 A1
(43) Pub. Date: Jul. 24, 2025

(54) CULTURE MEDIA FOR BACTERIA OF THE PASTEURELLACEAE FAMILY

(71) Applicant: Iowa State University Research Foundation, Inc., Ames, IA (US)

(72) Inventors: Mariela Elizabeth Srednik, Ames, IA (US); Qijing Zhang, Ames, IA (US); Mohamed El-Gazzar, Ames, IA (US); Yuko Sato, Ames, IA (US); Nubia Macedo, Ames, IA (US); Orhan Sahin, Ames, IA (US)

(21) Appl. No.: 19/034,934

(22) Filed: Jan. 23, 2025

Related U.S. Application Data
(60) Provisional application No. 63/623,913, filed on Jan. 23, 2024.

(51) Int. Cl. C12N 1/20

(52) U.S. Cl. CPC C12N 1/20

(57) ABSTRACT

The disclosure relates to bacterial culture media, useful for growing bacteria belonging to the Pasteurellaceae family, and methods of producing and using the described media.

### CROSS REFERENCE TO RELATED APPLICATIONS

This application claims priority to provisional application U.S. Ser. No. 63/623,913, filed Jan. 23, 2024, which is incorporated herein by reference in its entirety.

### TECHNICAL FIELD

The present disclosure relates to media compositions for culturing bacteria.

### BACKGROUND

Avibacterium paragallinarum (AP) is a fastidious, slow-growing organism, and is a major pathogen that causes infectious coryza, an economically important disease in layer chickens. For diagnosis, prevention, and control of the disease, isolation of the pathogen from diseased tissues is essential. However, AP is difficult to isolate and is often overgrown by other faster-growing commensals during the culturing process.

### SUMMARY

The need outlined above is met by the present disclosure which, in various embodiments, provides culture media that don't require nurse bacteria and also inhibit the growth of background commensal bacteria, leading to efficient isolation of pure AP or other bacteria of the Pasteurellaceae family.

Media compositions for culturing a bacterial species of the Pasteurellaceae family comprising a carbon source, a nitrogen source, serum, and nicotinamide adenine dinucleotide (NAD) are provided. In certain embodiments, the carbon source comprises starch. In certain embodiments, the nitrogen source comprises beef extract and casein hydrolysate. In certain embodiments, the serum is fetal bovine serum (FBS). In certain embodiments, the media compositions further comprise an inhibitor of Gram-positive bacteria such as vancomycin or crystal violet.

In certain embodiments, the media compositions comprise from about 1.5 g/L to about 2.5 g/L of beef extract; from about 15 g/L to about 20 g/L of casein hydrolysate; from about 1.2 g/L to about 1.8 g/L of starch; from about 4% to about 6% of FBS; from about 0.002% to about 0.003% of NAD; and from about 40 μg/mL to about 60 μg/mL of vancomycin or from about 3 μg/mL to about 5 μg/mL of crystal violet.

In one embodiment, the medium composition comprises about 2 g/L beef extract; about 17.5 g/L casein hydrolysate; about 1.5 g/L starch; about 5% FBS; about 0.0025% NAD; about 50 g/mL vancomycin, and optionally about 17 g/L agar.

In another embodiment, the medium composition comprises about 2 g/L beef extract; about 17.5 g/L casein hydrolysate; about 1.5 g/L starch; about 5% FBS; about 0.0025% NAD; about 4 μg/mL crystal violet, and optionally about 17 g/L agar.

Methods of isolating and/or culturing a bacterial species of the Pasteurellaceae family comprising inoculating a sample into a medium composition of the disclosure; and incubating the inoculated media for a period of time sufficient to permit growth of the bacteria are also provided. In certain embodiments, the sample is from a chicken and the bacterial species of the Pasteurellaceae family is Avibacterium paragallinarum.

Methods for the growth of a bacterial species of the Pasteurellaceae family as a pure immunogen for vaccine preparation comprising inoculating a seed strain into a medium composition of the disclosure are also provided.

While multiple embodiments are disclosed, still other embodiments of the present disclosure will become apparent based on the detailed description, which shows and describes illustrative embodiments of the disclosure. Accordingly, the figures and detailed description are to be regarded as illustrative in nature and not restrictive.

### BRIEF DESCRIPTION OF THE FIGURES

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1 shows AP cultured on chocolate agar plus nurse, MSNV, and MSCV agar.

FIG. 2A-C shows evaluation of different concentrations of vancomycin and crystal violet for inhibition of commensal bacteria. FIG. 2A shows MSNV: 5 g/mL of vancomycin was enough to inhibit Staphylococcus spp., a common inhabitant in chicken tissue samples, but even the highest concentration was not enough to inhibit Lactobacillus salivarius, another frequent contaminant in clinical samples. FIG. 2B shows MSCV: 0.5 μg/mL of crystal violet was enough to inhibit Staphylococcus spp., while 4 μg/mL of crystal violet was needed to inhibit Lactobacillus salivarius, a frequent contaminant in samples. In both FIG. 2A and FIG. 2B, AP was able to grow at all tested concentrations of vancomycin and crystal violet. FIG. 2C shows inhibition of Gram-positive bacteria by 4 μg/ml crystal violet in a mixed culture with AP.

FIG. 3 shows the ability of MSNV and MSCV to support the growth of different genera of the Pasteurellaceae family in comparison with chocolate agar.

FIG. 4A-B shows a comparison of MSNV and MSCV with chocolate agar in isolation of AP from clinical samples of chicken infectious coryza. FIG. 4A shows AP infected tissue lacking background commensals. FIG. 4B shows AP infected tissue with background commensal bacteria on chocolate agar, but not on MSNV and MSCV.

FIG. 5 shows MSCV media with the addition of crystal violet helped to inhibit Lactobacillus salivarius, a frequent contaminant in chicken clinical samples, and also helps to differentiate A. paragallinarum colonies (light violet) from another bacterial genera.

### DETAILED DESCRIPTION

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various embodiments of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The culture media according to the present disclosure are designed to be suitable to grow or maintain/support the growth of one or more bacterial species belonging to the Pasteurellaceae family. The culture media according to the present disclosure typically comprise a carbon source, a nitrogen source, serum, optionally a cofactor, optionally an inhibitor of Gram-positive bacteria, and optionally a solidification component.

The culture media according to the present disclosure comprises a carbon source. Examples of suitable carbon sources which may be used for growing bacteria include, but are not limited to, monosaccharides such as glucose, fructose, and the like; disaccharides such as maltose, sucrose, and the like; oligosaccharides; polysaccharides such as dextrin, cyclodextrin, starch, and the like; sugar alcohols such as xylitol, sorbitol, erythritol, and the like; salts of organic acids such as acetic acid, fumaric acid, adipic acid, propionic acid, citric acid, gluconic acid, malic acid, pyruvic acid, malonic acid and the like; alcohols such as ethanol and glycerol and the like; oil or fat such as soybean oil, rice bran oil, olive oil, corn oil, sesame oil. In certain embodiments, the carbon source comprises starch.

In certain embodiments, the medium comprises about 0.1 g/L to about 3 g/L of a carbon source, about 0.5 g/L to about 2 g/L of a carbon source, about 1.2 g/L to about 1.8 g/L of a carbon source, or about 1.4 g/L to about 1.6 g/L of a carbon source. The concentration may be any value or subrange within the recited ranges, including endpoints. For example, the medium can include about 0.5 g/L of a carbon source, about 1 g/L of a carbon source, about 1.5 g/L of a carbon source, about 2 g/L of a carbon source, about 2.5 g/L of a carbon source, or about 3 g/L of a carbon source.

In certain embodiments, the carbon source is starch. In certain embodiments, the medium comprises about 0.1 g/L to about 3 g/L starch, about 0.5 g/L to about 2 g/L starch, about 1.2 g/L to about 1.8 g/L starch, or about 1.4 g/L to about 1.6 g/L of a carbon source. The concentration may be any value or subrange within the recited ranges, including endpoints. For example, the medium can include about 0.5 g/L starch, about 1 g/L starch, about 1.5 g/L starch, about 2 g/L starch, about 2.5 g/L starch, or about 3 g/L starch.

Examples of suitable nitrogen sources which may be used for growing bacteria include, but are not limited to, nonfat dry milk, whey protein, yeast extract, malt extract, beef extract, casein hydrolysate, tryptone, cysteine, peptone, amino acids, potassium nitrate, ammonium nitrate, ammonium chloride, ammonium sulfate, ammonium phosphate, and ammonia. In certain embodiments, the nitrogen source comprises beef extract. In certain embodiments, the nitrogen source comprises casein hydrolysate. In certain embodiments, the nitrogen source comprises beef extract and casein hydrolysate.

In certain embodiments, the medium comprises about 1 g/L to about 40 g/L of a nitrogen source, about 10 g/L to about 30 g/L of a nitrogen source, about 15 g/L to about 25 g/L of a nitrogen source, or about 18 g/L to about 22 g/L of a nitrogen source. The concentration may be any value or subrange within the recited ranges, including endpoints. For example, the medium can include about 1 g/L of a nitrogen source, about 5 g/L of a nitrogen source, about 10 g/L of a nitrogen source, about 15 g/L of a nitrogen source, about 20 g/L of a nitrogen source, about 25 g/L of a nitrogen source, about 30 g/L of a nitrogen source, about 35 g/L of a nitrogen source, or about 40 g/L of a nitrogen source.

In certain embodiments, the nitrogen source is beef extract. In certain embodiments, the medium comprises about 0.1 g/L to about 4 g/L beef extract, about 1 g/L to about 3 g/L beef extract, about 1.5 g/L to about 2.5 g/L beef extract, or about 1.8 g/L to about 2.2 g/L beef extract. The concentration may be any value or subrange within the recited ranges, including endpoints. For example, the medium can include about 0.5 g/L beef extract, about 1 g/L beef extract, about 1.5 g/L beef extract, about 2 g/L beef extract, about 2.5 g/L beef extract, about 3 g/L beef extract, about 3.5 g/L beef extract, or about 4 g/L beef extract.

In certain embodiments, the nitrogen source is casein hydrolysate. In certain embodiments, the medium comprises about 1 g/L to about 35 g/L casein hydrolysate, about 5 g/L to about 30 g/L casein hydrolysate, or about 15 g/L to about 20 g/L casein hydrolysate. The concentration may be any value or subrange within the recited ranges, including endpoints. For example, the medium can include about 1 g/L casein hydrolysate, about 5 g/L casein hydrolysate, about 10 g/L casein hydrolysate, about 15 g/L casein hydrolysate, about 20 g/L casein hydrolysate, about 25 g/L casein hydrolysate, about 30 g/L casein hydrolysate, or about 35 g/L casein hydrolysate.

Any serum suitable for use in a culture medium may be comprised within a culture medium according to the present disclosure. In certain embodiments, the serum is a synthetic serum. A synthetic serum is typically a serum which has been manufactured using component parts present in serum (electrolytes, antibodies, antigens and hormones). In certain embodiments, the serum is an animal serum. In certain embodiments, the serum is one or more selected from horse serum, sheep serum, goat serum, bovine serum, fetal bovine serum, calf serum, mouse serum, rat serum, pig serum, guinea pig serum, porcine serum, chicken serum, and rabbit serum. In certain embodiments, the serum is fetal bovine serum (FBS).

In certain embodiments, the medium comprises about 0.5% to about 10% serum (e.g., fetal bovine serum), about 2.5% to about 7.5% serum, or about 4% to about 6% serum. The concentration may be any value or subrange within the recited ranges, including endpoints. For example, the medium can include about 1% serum (e.g., fetal bovine serum), about 2% serum, about 3% serum, about 4% serum, about 5% serum, about 6% serum, about 7% serum, about 8% serum, about 9% serum, or about 10% serum.

Examples of cofactors include thiamine derivatives, biotin, vitamin C, NAD/NADP, cobalamin, flavin mononucleotide and derivatives, glutathione, heme nucleotide phosphates, and derivatives.

In certain embodiments, the cofactor is NAD. In certain embodiments, the medium comprises about 0.0001% to about 0.005% NAD, about 0.001% to about 0.004% NAD, or about 0.002% to about 0.003% NAD. The concentration may be any value or subrange within the recited ranges, including endpoints. For example, the medium can include about 0.001% NAD, about 0.0015% NAD, about 0.002% NAD, about 0.0025% NAD, about 0.003% NAD, about 0.0035% NAD, about 0.004% NAD, about 0.0045% NAD, or about 0.005% NAD.

Either NAD or its reduced form (NADH) is necessary for the in vitro growth of most AP isolates, although there are exceptions where the isolates are NAD independent (isolates from South Africa, Mexico and Peru). Due to the need for NAD/NADH for growth, a number of bacteria excreting V-factor (which comprises NAD) are typically used as nurse bacteria to support growth of AP on culture media, where AP demonstrates satellite growth around the nurse bacteria. Staphylococcus aureus, Staphylococcus hyicus, and Staphylococcus chromogenes have been demonstrated to be good nurses for AP growth. In contrast, the media compositions of the present disclosure do require a nurse bacterium to support growth.

The media compositions of the disclosure may further comprise an inhibitor of Gram-positive bacteria to prevent the overgrowth of background commensals. Examples of suitable inhibitors of Gram-positive bacteria which may be used in the media compositions include, but are not limited to, vancomycin and crystal violet.

In certain embodiments, the inhibitor of Gram-positive bacteria is vancomycin. In certain embodiments, the medium comprises about 5 μg/mL to about 100 μg/mL vancomycin, about 25 μg/mL to about 75 μg/mL vancomycin, or about 40 μg/mL to about 60 μg/mL vancomycin. The concentration may be any value or subrange within the recited ranges, including endpoints. For example, the medium can include about 10 μg/mL vancomycin, about 20 μg/mL vancomycin, about 30 μg/mL vancomycin, about 40 g/mL vancomycin, about 50 μg/mL vancomycin, about 60 μg/mL vancomycin, about 70 μg/mL vancomycin, about 80 μg/mL vancomycin, about 90 μg/mL vancomycin, or about 1000 μg/mL vancomycin.

In certain embodiments, the inhibitor of Gram-positive bacteria is crystal violet. In certain embodiments, the medium comprises about 0.5 g/mL to about 10 μg/mL crystal violet, about 2 μg/mL to about 6 μg/mL crystal violet, or about 3 μg/mL to about 5 μg/mL crystal violet. The concentration may be any value or subrange within the recited ranges, including endpoints. For example, the medium can include about 1 μg/mL crystal violet, about 2 μg/mL crystal violet, about 3 μg/mL crystal violet, about 4 μg/mL crystal violet, about 5 μg/mL crystal violet, about 6 μg/mL crystal violet, about 7 μg/mL crystal violet, about 8 μg/mL crystal violet, about 9 μg/mL crystal violet, or about 10 μg/mL crystal violet.

The media of the disclosure may be in a liquid form, i.e., a broth, or may be in solid or semi-solid form, e.g., agar-based. A solid or semi-solid medium contains a solidification component for keeping the medium solid. Examples of a solidification component include agar, gelatin, collagen, gellan gum, and the like. Of these, agar is preferred. The solidification components may be used singly, or in a combination of two or more.

The concentration of the solidification component in the solid or semi-solid medium is not particularly limited, and may vary depending on the type of the solidification component. The concentration may be, for example, about 1 g/L to about 50 g/L, about 10 g/L to about 25 g/L, or about 15 g/L to about 20 g/L. The concentration may be any value or subrange within the recited ranges, including endpoints. For example, the solid or semi-solid medium can include about 10 g/L, about 11 g/L, about 12 g/L, about 13 g/L, about 14 g/L, about 15 g/L, about 16 g/L, about 17 g/L, about 18 g/L, about 19 g/L, or about 20 g/L of the solidification component (e.g., agar).

Methods of isolating and/or culturing a bacterial species of the Pasteurellaceae family are provided. Pasteurellaceae comprise a large and diverse family of Gram-negative proteobacteria comprising the genera Actinobacillus, Aggregatibacter, Avibacterium, Basfia, Bibersteinia, Chelonobacter, Gallibacterium, Haemophilus, Histophilus, Lonepinella, Mannheimia, Nicoletella, Pasteurella, Phocoenobacter and Volucribater. Some species in these genera are important human or animal pathogens, e.g., Haemophilus influenzae or Mannheimia haemolytica, while others are commensals of the animal and human mucosa, mostly in the upper respiratory tract. H. influenzae causes several respiratory diseases in humans and is also known as an agent of meningitis in children. Other Pasteurellaceae cause gingivitis and chancroid in humans and many others are important veterinary pathogens, e.g., Avibacterium paragallinarum, the cause of infectious coryza in chicken; Mannheimia haemolytica, the cause of bronchopneumonia in cattle; Actinobacillus pleuropneumoniae, a responsible agent of pneumonia in swine; or Haemophilus parasuis, causing severe polyserositis in pigs.

The cell culture may be performed in any container suitable for the culture of cells, such as a petri dish, contact plate, bottle, tube, well, vessel, bag, flask or tank. Typically the container is sterilized prior to use. Incubation is typically performed under suitable conditions such as suitable temperature, osmolarity, aeration, agitation, etc. A person skilled in the art is aware of suitable incubation conditions for supporting or maintaining the growth/culturing of cells.

Methods for the culture of bacterial cells are well known to those skilled in the art. For example, when a solid or semi-solid medium is employed, a method in which bacterial cells are plated on the surface of the medium and cultured under conditions suitable for culture is used.

Ambient conditions suitable for cell culture, such as temperature and atmospheric composition, are also well known to those skilled in the art.

The culture temperature is not particularly limited, as long as the growth of the bacteria is not significantly inhibited. The culture temperature may be, for example, about 25° C. to about 45° C. or about 34° C. to about 42° C. The temperature may be any value or subrange within the recited ranges, including endpoints. For example, the temperature may be about 34° C., about 35° C., about 36° C., about 37° C., about 38° C., about 39° C., about 40° C., about 41° C., or about 42° C.

The culture atmosphere is not particularly limited, as long as the growth of the bacteria is not significantly inhibited. Examples of the culture atmosphere include air, an atmosphere in which carbon dioxide is introduced into the atmosphere to increase the concentration thereof (e.g., 5% to 7.5% carbon dioxide), and the like.

The culture time is not particularly limited, as long as the growth at a certain level is possible. The culture time may be, for example, about 30 minutes to about 48 hours, about 1 hour to about 24 hours, or about 2 about to about 12 hours.

The following numbered embodiments also form part of the present disclosure:
1. A medium composition for culturing a bacterial species of the Pasteurellaceae family, the composition comprising: a carbon source; a nitrogen source; serum; nicotinamide adenine dinucleotide (NAD).
2. The medium composition of embodiment 1, wherein the nitrogen source comprises from about 0.1 g/L to about 4 g/L of beef extract and from about 1 g/L to about 35 g/L of casein hydrolysate.
3. The medium composition of embodiment 1 or embodiment 2, wherein the carbon source comprises from about 0.1 g/L to about 3 g/L of starch.
4. The medium composition of any one of embodiments 1-3, wherein the serum is fetal bovine serum (FBS).
5. The medium composition of any one of embodiments 1-4, wherein the composition comprises from about 0.5% to about 10% of FBS.
6. The medium composition of any one of embodiments 1-5, wherein the composition comprises from about 0.0001% to about 0.005% NAD.
7. The medium composition of any one of embodiments 1-6, further comprising an inhibitor of Gram-positive bacteria.
8. The medium composition of any one of embodiments 1-7, wherein the inhibitor of Gram-positive bacteria comprises vancomycin or crystal violet.
9. The medium composition of any one of embodiments 1-8, wherein the composition comprises: from about 1.5 g/L to about 2.5 g/L of beef extract; from about 15 g/L to about 20 g/L of casein hydrolysate; from about 1.2 g/L to about 1.8 g/L of starch; from about 4% to about 6% of FBS; from about 0.002% to about 0.003% of NAD; and from about 40 g/mL to about 60 μg/mL of vancomycin or from about 3 μg/mL to about 5 μg/mL of crystal violet.
10. The medium composition of any one of embodiments 1-9, further comprising a solidification component, optionally wherein the solidification component is agar.
11. The medium composition of any one of embodiments 1-10, wherein the composition has a pH from about 6 to about 8.
12. A method of isolating and/or culturing a bacterial species of the Pasteurellaceae family, the method comprising: inoculating a sample into the medium composition of any one of embodiments 1-11; and incubating the inoculated medium for a period of time sufficient to permit growth of the bacteria.
13. The method of embodiment 12, wherein the medium composition is not inoculated with a nurse bacterium.
14. The method of embodiment 12 or embodiment 13, wherein the sample is from chicken, pig, or cattle.
15. The method of any one of embodiments 12-14, wherein the bacterial species of the Pasteurellaceae family is selected from Actinobacillus spp., Avibacterium spp., Glaesserella, spp., Haemophilus spp., Mannheimia spp., or Pasteurella spp.
16. The method of any one of embodiments 12-15, wherein the bacterial species of the Pasteurellaceae family is Avibacterium paragallinarum.
17. The method of any one of embodiments 12-16, wherein the inoculated medium is incubated at a carbon dioxide concentration of about 5% to about 7.5%.
18. The method of any one of embodiments 12-17, wherein the inoculated medium is incubated at a temperature of about 34° C. to about 42° C.
19. A method for the growth of a bacterial species of the Pasteurellaceae family as a pure immunogen for vaccine preparation comprising: inoculating a seed strain into the medium composition of any one of embodiments 1-11; and incubating the inoculated medium for a period of time sufficient to permit growth of the bacteria.
20. The method of embodiment 19, wherein the medium composition is not inoculated with a nurse bacterium.
21. The method of embodiment 19 or embodiment 20, wherein the bacterial species of the Pasteurellaceae family is selected from Actinobacillus spp., Avibacterium spp., Glaesserella, spp., Haemophilus spp., Mannheimia spp., or Pasteurella spp.
22. The method of any one of embodiments 19-21, wherein the bacterial species of the Pasteurellaceae family is Avibacterium paragallinarum.
23. The method of any one of embodiments 19-22, wherein the inoculated medium is incubated at a carbon dioxide concentration of about 5% to about 7.5%.
24. The method of any one of embodiments 19-23, wherein the inoculated medium is incubated at a temperature of about 34° C. to about 42° C.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended claims.

The following examples are offered by way of illustration and not by way of limitation.

### EXAMPLES

### Example 1: Development of an Improved Culture Media for Isolation of AP from Clinical Samples

Several different media are used for the isolation of AP from chicken infraorbital sinus in research and diagnostic laboratories (TABLE 1). Most of the media require the use of nurse bacteria. For example, the Veterinary Diagnostic Laboratory at Iowa State University (ISU VDL) currently utilizes blood agar and chocolate agar with the addition of a nurse bacterium (Staphylococcus hyicus) to isolate AP. The use of a nurse bacterium doesn't prevent the growth of commensal bacteria from chicken tissues and also generates a high risk for contamination of the pure isolates. There were reported methods that utilize brain heart infusion (BI) agar supplemented with NAD for isolation of AP. Although this culture medium eliminates need for nurse bacteria, it doesn't prevent the overgrowth of background commensals and is inefficient for AP isolation.

TABLE 1

| Authors | Year | Media |
|---|---|---|
| Rimler R B | 1979 | Test-medium agar contained 1% Biosate, 1% sodium chloride, |
| | | 0.1% soluble starch, 0.05% dextrose, 0.005% thiamine-HCl, |
| | | 1.5% Noble agar |
| Blackall P J et al. | 1982 | Test medium agar supplemented with 5% (v/v) oleic albumin |
| | | complex, 1% (v/v) filter sterilized, heat inactivated chicken |
| | | serum and 0.0025% (w/v) NADH. |
| Eaves L E et al. | 1989 | TM/SN medium: 1% Biosate peptone, 1% NaCl, 0.1% starch, |
| | | 0.05% glucose, 1.5% Noble agar base supplemented with 5% |
| | | (v/v) oleic albumin complex, 0.0005% (v/v) thiamine, 1% (v/v) |
| | | heart-inactivated chicken serum, 0.003% (w/v) NADH. |
| Horner R F et al. | 1995 | Chocolate Columbia blood agar supplemented with 50 ug/ml NAD. |
| Terzolo H R et al. | 1993 | Columbia blood agar base with 7% equine lysed blood with |
| | | bacitracin, cloxacillin and vancomycin. |
| Terzolo H R et al. | 1997 | Balcarce broth: 1 g proteose-peptone, 1 g tryptose, 0.3 g meat |
| | | extract, 0.05 g dextrose, 0.1 g soluble starch, 0.5 g sodium chloride, |
| | | and water 100 ml. Supplemented with 25 ug NADH/ml and 1% |
| | | (v/v) inactivated horse serum. |
| Fernandez R P et al. | 2005 | 10% sheep blood agar with S. epidermidis as colony feeder. |
| | | Brain-heart infusion broth supplemented with 1% (v/v) sodium |
| | | chloride, 0.0025% (w/v) NADH, and 1% (v/v) filter-sterilized, |
| | | heart-inactivated horse serum. |
| Mendoza-Espinosa A | 2009 | GC agar, supplemented with 25 ug/ml of NADH, 5% inactivated |
| et al. | | chicken serum, and 1% Iso VitaleX ™ enrichment. |
| Sharmin A et al. | 2014 | Blood agar with NAD, blood agar with feeder colony of S. aureus, |
| | | Chocolate agar with NAD, Chocolate agar with feeder colony |
| | | (S. aureus). |
| Liu C C et al. | 2016 | Brain-heart infusion agar supplemented with 5% bovine serum |
| | | and 0.01% NAD |
| Jeong O M et al. | 2017 | Test Medium Agar supplemented with 5% (v/v) oleic albumin |
| | | complex, 1% (v/v) chicken serum, 0.0025% (w/v) NADH and |
| | | 0.005% thiamine-HCL. |
| Feberwee A et al. | 2019 | Chocolate agar enriched with NAD and 6% sheep blood. |
| Caballero-García M | 2020 | Columbia agar plus 7% equine hemolyzed blood with or without |
| et al. | | the addition of antibiotics (bacitracin, cloxacillin, or vancomycin). |
| Guo M et al. | 2021 | Trypticase soy agar supplemented with 10% FBS and |
| | | 0.0025% NADH. |
| Wu Y et al. | 2021 | Tryptic soy agar supplemented with 5% (v/v) FBS and |
| | | 0.001% (w/v) NAD. |
| Beiranvand S et al. | 2022 | BHI broth enriched with 0.0025 mg/ml NAD. |

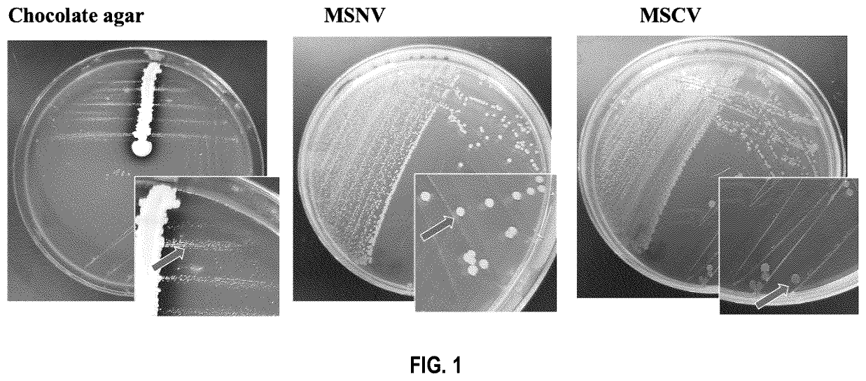

To develop better culture media for isolation of AP from clinical samples, different base media including Mueller-Hinton agar (MHA), BI, and Columbia agar supplemented with different growth factors were compared in the presence or absence of nurse bacteria for growth of AP. Various media that were tested are summarized in TABLE 2. In the absence of a nurse bacterium, MHA plus 5% fetal bovine serum (FBS) and 0.0025% NAD demonstrated the best growth of AP as evidenced by appearance of bacterial colonies on the full plate (FIG. 1).

TABLE 2
Media

Tryptic soy agar base (TS) + sheep blood + NAD
TS + hemolyzed sheep blood + NAD
TS + laked horse blood + NAD
Casman medium (CM) + equine hemolyzed blood (EHB)
Columbia agar base (CA) + EHB
Columbia blood agar (CBA) + EHB
CM + fetal bovine serum (FBS) + NAD
CA + FBS + NAD
CBA + FBS + NAD
Mueller Hinton agar (MHA) + FBS + NAD
MHA + FBS + S. chromogenes supernatant

The addition of vancomycin (MSNV) or crystal violet (MSCV) as inhibitors of Gram-positive commensals did not affect the growth of AP but significantly improved the isolation of AP from clinical samples (FIG. 1 and TABLE 3). One of the most significant improvements was the number and size of colonies that can be isolated using these two media in comparison with Chocolate or blood agar plus nurse. Although colonies grow in all the media, colony sizes in MSNV and MSCV are much bigger than in Chocolate agar that appear as dewdrops.

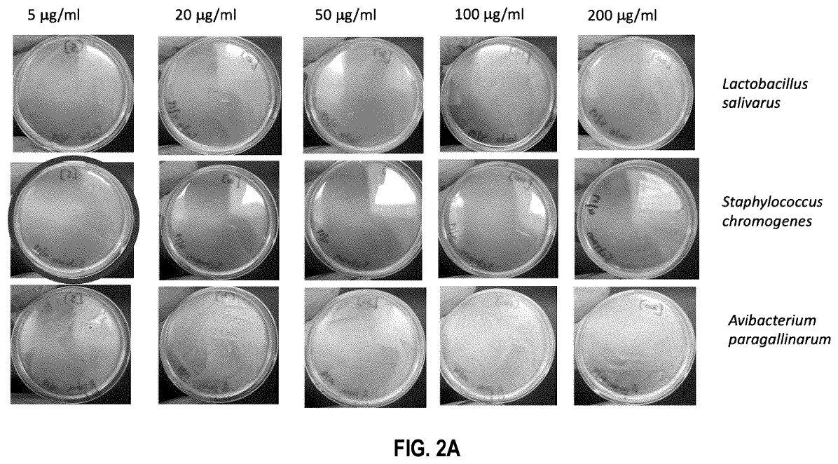

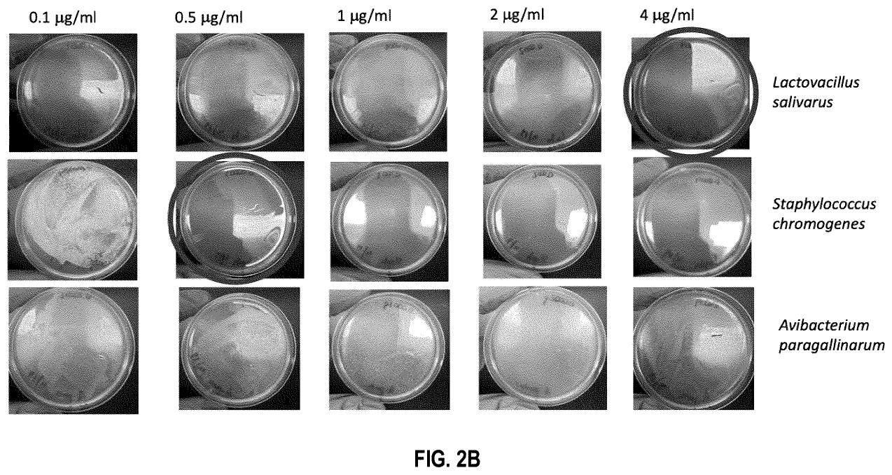

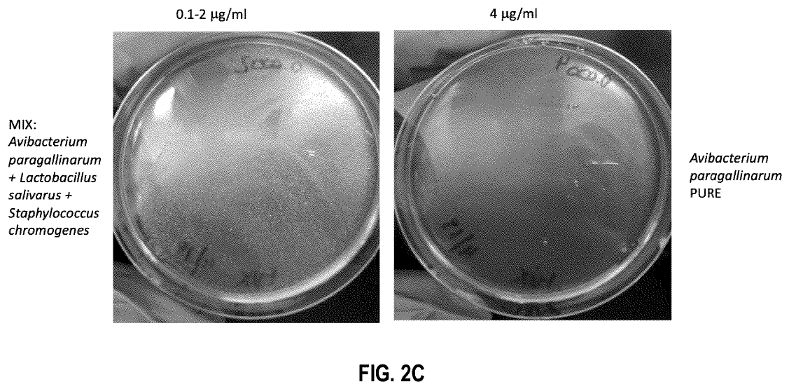

Additionally, different concentration of vancomycin and crystal violet were tested against a mixture of bacterial cultures (Avibacterium paragallinarum, Staphylococcus spp., and Lactobacillus salivarius) to determine the optimal concentrations for inhibiting background bacteria (FIG. 2). Vancomycin is an antibiotic that is commonly used in culture media to inhibit Gram positive bacteria. With vancomycin, Staphylococcus spp. that is often present in the mucosal and skin surfaces was inhibited at a concentration of 5 μg/mL. However, Lactobacillus salivarius, which is a frequent contaminant in clinical samples, was not inhibited by vancomycin even at the 200 μg/mL vancomycin concentration, but was inhibited with 4 μg/mL of crystal violet. Based on the results, use of 50 μg/mL vancomycin and 4 μg/mL crystal violet for the MSNV and MSCV media, respectively, was proposed.

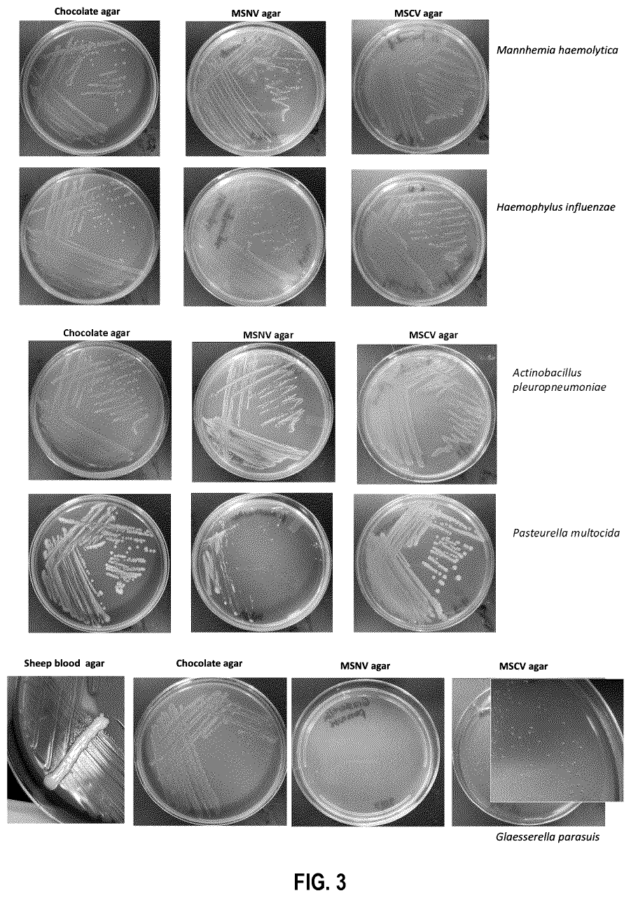

Other genera members of the Pasteurellaceae family (Pasteurella, Actinobacillus, Haemophilus, Mannheimia, Glaesserella) were also cultured. All genera members grew very well in the MSCV media, but in the MSNV media, all except one member (Glaesserella parasuis) showed to be resistant to vancomycin (FIG. 3). Since Pasteurellaceae family encompasses genus and species that infect different animal species, the media can be a potentially used to isolate other bacterial pathogens from different animal species.

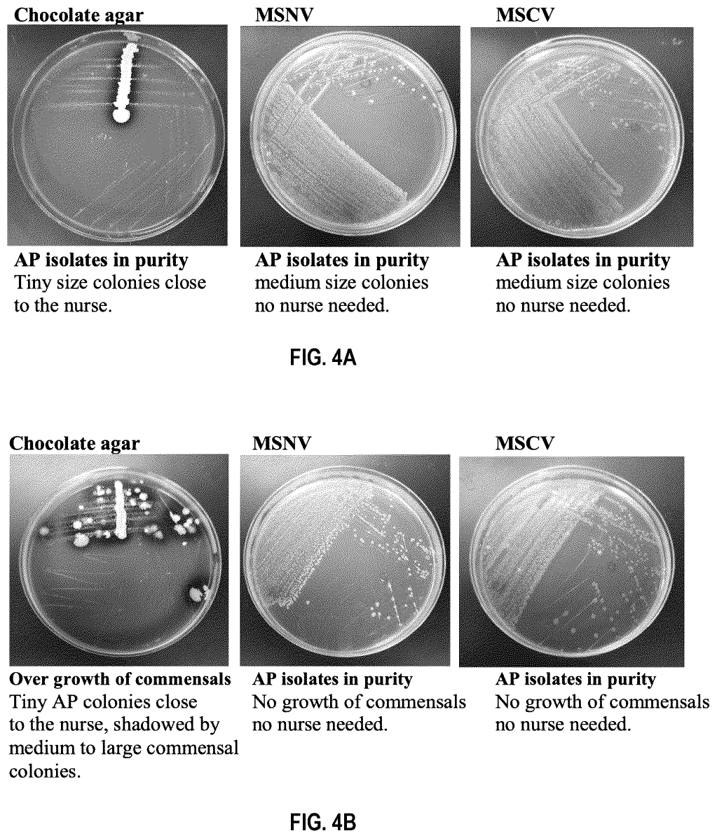

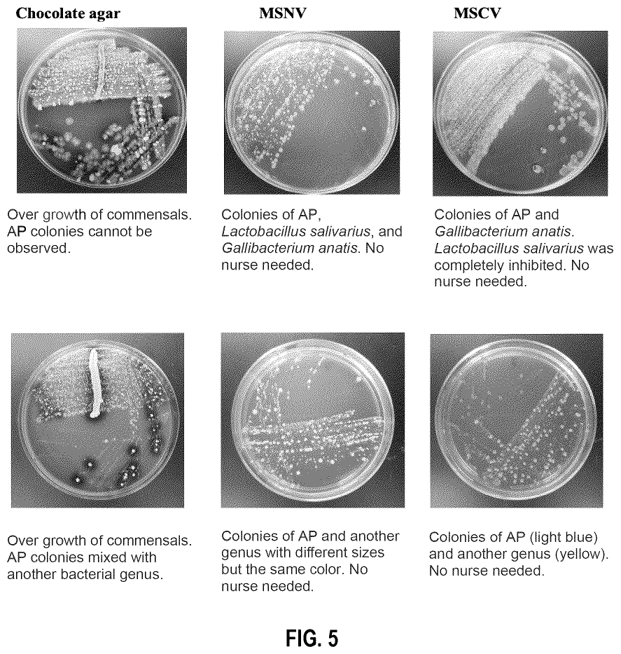

When these two media were used for isolation of AP from clinical samples, larger AP colonies were observed in purity than using the conventional agar plates (FIG. 4). When the samples were contaminated by Gram negative bacteria, AP could be differentiated from other colonies based on size and morphology, and with the MSCV media AP colonies showed a light violet color, facilitating its differentiation from others (FIG. 5).

After using these two media side-by-side with the same clinical samples, the isolation rates of AP and Avibacterium spp. were significantly improved. In addition, non-typical AP were able to be isolated, which the conventional culture plates were not able (TABLE 3). In conclusion, with the use of these two media, the A. paragallinarum isolation from clinical samples and from chicken samples without clinical symptoms was considerably improved.

TABLE 3

| | Coryza | | Results from the | |
|---|---|---|---|---|
| Cases* | Symptoms | qPCR** | conventional media | Results from MSNV/MSCV |
| 207 chicken head | + | + | N = 78 A. paragallinarum | N = 131 A. paragallinarum |
| samples | | | N = 15 Avibacterium spp | N = 18 Avibacterium spp |
| 153 chicken head | − | + | N = 1 Avibacterium spp | N = 4 A. paragallinarum |
| samples | | | | N = 6 Avibacterium spp |
| 5 chicken head | + | − | N = 3 Avibacterium spp | N = 2 Avibacterium spp |
| samples | | | | |
| 35 chicken head | − | − | N = 0 | N = 11 Avibacterium spp |
| samples | | | | |
| Total: 400 samples | | | N = 78 A. paragallinarum | N = 135 A. paragallinarum |
| | | | N = 15 Avibacterium spp | N = 23 Avibacterium spp |

*Number of chicken head samples received at ISU VDL from chickens with or without Coryza symptoms
**Results of qPCR detection for Avibacterium paragallinarum

What is claimed is:

1. A medium composition for culturing a bacterial species of the Pasteurellaceae family, the composition comprising: a carbon source; a nitrogen source; serum; nicotinamide adenine dinucleotide (NAD).

2. The medium composition of claim 1, wherein the nitrogen source comprises from about 0.1 g/L to about 4 g/L of beef extract and from about 1 g/L to about 35 g/L of casein hydrolysate.

3. The medium composition of claim 1, wherein the carbon source comprises from about 0.1 g/L to about 3 g/L of starch.

4. The medium composition of claim 1, wherein the serum is fetal bovine serum (FBS).

5. The medium composition of claim 4, wherein the composition comprises from about 0.5% to about 10% of FBS.

6. The medium composition of claim 1, wherein the composition comprises from about 0.0001% to about 0.005% NAD.

7. The medium composition of claim 1, further comprising an inhibitor of Gram-positive bacteria.

8. The medium composition of claim 7, wherein the inhibitor of Gram-positive bacteria comprises vancomycin or crystal violet.

9. The medium composition of claim 1, wherein the composition comprises:
from about 1.5 g/L to about 2.5 g/L of beef extract;
from about 15 g/L to about 20 g/L of casein hydrolysate;
from about 1.2 g/L to about 1.8 g/L of starch;
from about 4% to about 6% of FBS;
from about 0.002% to about 0.003% of NAD; and
from about 40 μg/mL to about 60 μg/mL of vancomycin or from about 3 μg/mL to about 5 μg/mL of crystal violet.

10. The medium of claim 1, further comprising a solidification component.

11. The medium of claim 1, wherein the composition has a pH from about 6 to about 8.

12. A method of isolating and/or culturing a bacterial species of the Pasteurellaceae family, the method comprising: inoculating a sample into the medium composition of claim 1; and
incubating the inoculated medium for a period of time sufficient to permit growth of the bacteria.

13. The method of claim 12, wherein the medium composition is not inoculated with a nurse bacterium.

14. The method of claim 12, wherein the sample is from chicken, pig, or cattle.

15. The method of claim 12, wherein the bacterial species of the Pasteurellaceae family is selected from Actinobacillus spp., Avibacterium spp., Glaesserella, spp., Haemophilus spp., Mannheimia spp., or Pasteurella spp.

16. The method of claim 12, wherein the bacterial species of the Pasteurellaceae family is Avibacterium paragallinarum.

17. The method of claim 12, wherein the inoculated medium is incubated at a carbon dioxide concentration of about 5% to about 7.5%.

18. The method of claim 12, wherein the inoculated medium is incubated at a temperature of about 34° C. to about 42° C.

19. The method of claim 12, wherein the composition comprises:
from about 1.5 g/L to about 2.5 g/L of beef extract;
from about 15 g/L to about 20 g/L of casein hydrolysate;
from about 1.2 g/L to about 1.8 g/L of starch;
from about 4% to about 6% of FBS;
from about 0.002% to about 0.003% of NAD; and
from about 40 μg/mL to about 60 μg/mL of vancomycin or from about 3 μg/mL to about 5 μg/mL of crystal violet.

20. A method for the growth of a bacterial species of the Pasteurellaceae family as a pure immunogen for vaccine preparation comprising: inoculating a seed strain into the medium composition of claim 1; and incubating the inoculated medium for a period of time sufficient to permit growth of the bacteria.
